(19) United States
(12) Patent Application Publication
(10) Pub. No.: US 2004/0210232 A1
(43) Pub. Date: Oct. 21, 2004

(54) Guide device and plate inserter

(76) Inventors: Tushar Patel, Potomac, MD (US); Eric D. Kolb, Milton, MA (US); Jonathan Fanger, Fall River, MA (US); Richard Fessler, Winnetka, IL (US); Alan Crockard, London (GB); Todd Albert, Penn Valley, PA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/776,666

(22) Filed: Feb. 11, 2004

Related U.S. Application Data
(63) Continuation-in-part of application No. 10/409,958, filed on Apr. 9, 2003.

(51) Int. Cl. A61B 17/60

(52) U.S. Cl. 606/96

(57) ABSTRACT

A guide device for use in securing a spinal fixation plate to a spine is provided. In a preferred embodiment, the guide device generally includes a support member having first and second arms mated thereto. Each arm includes a proximal end and a distal end having at least one guide member coupled thereto and defining a pathway for receiving a tool. One or both arms can be slidably movable along the support member to allow the distance between the arms to be adjusted. In use, the arms can be adjusted to couple to a spinal fixation plate and to position the guide member on each arm in alignment with the fixation plate. The guide device can then be used to drill, awl, tap, and insert implants, such as spinal screws, into the vertebral bodies to attach the fixation plate thereto.

### CROSS-REFERENCE TO RELATED APPLICATIONS

This application is a continuation-in-part of U.S. patent application Ser. No. 10/409,958, filed on Apr. 9, 2003, entitled “Drill Guide and Plate Inserter,” which is expressly incorporated herein by reference.

### FIELD OF THE INVENTION

The present invention relates to devices for assisting in spinal surgery, and more particularly to a guide device and plate inserter for introducing spinal tools and devices.

### BACKGROUND OF THE INVENTION

Advancing age, as well as injury, can lead to changes in the bones, discs, joints, and ligaments of the spine, producing pain from nerve root compression. Under certain circumstances, alleviation of pain can be provided by performing a spinal fusion. This is a procedure that involves joining two or more adjacent vertebrae with a bone fixation device so that they no longer are able to move relative to each other. For a number of known reasons, bone fixation devices are useful for promoting proper healing of injured or damaged vertebral bone segments caused by trauma, tumor growth, or degenerative disc disease. The external fixation devices immobilize the injured bone segments to ensure the proper growth of new osseous tissue between the damaged segments. These types of external bone fixation devices often include internal bracing and instrumentation to stabilize the spinal column to facilitate the efficient healing of the damaged area without deformity or instability, while minimizing any immobilization and post-operative care of the patient.

One such device is a bone fixation plate that is used to immobilize adjacent skeletal parts such as bones. Typically, the fixation plate is a rigid metal or polymeric plate positioned to span bones or bone segments that require immobilization with respect to one another. The plate is fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed.

Such plates have been used to immobilize a variety of bones, including vertebral bodies of the spine. These bone plate systems usually include a rigid bone plate having a plurality of screw openings. The openings are either holes or slots to allow for freedom of screw movement. The bone plate is placed against the damaged vertebral bodies and bone screws are used to secure the bone plate to the spine and optionally to a prosthetic implant or bone graft positioned between the adjacent vertebrae. Implantation of the plate, however, can be difficult. Each plate must be properly aligned with the vertebral bodies, and holes for receiving the bone screws must be drilled into the vertebrae at precise angles. It is often necessary to use the bone plate as a drill guide for drilling and tapping the bone in preparation for receiving the bone screws. Such a procedure can be difficult, however, as the surgeon is required to securely and rigidly hold the bone plate against the vertebrae, obtain proper alignment, drill, tap, and finally set the bone screws.

Accordingly, there remains a need for a guide instrument which can be used to assist in fastening a plate to a patient's spine.

### SUMMARY OF THE INVENTION

The present invention generally provides a guide device having a support member with first and second arms mated thereto. Each arm has a proximal end coupled to the elongate support member and a distal end having at least one guide member formed thereon. At least one of the guide members preferably includes a pathway formed therethrough for receiving a tool, but in an exemplary embodiment each guide member includes two pathways formed therethrough. The pathways can be formed within a housing having a variety of configurations, and they can be formed within separate lumens, or they can be at least partially in communication with one another. In use, the guide member is adapted to be juxtaposition on a spinal implant such that the pathways align with corresponding bores formed in the implant.

In one embodiment of the present invention, one or both of the first and second arms can be slidably movable along the support member to allow a distance between the first and second arms to be adjusted. In an exemplary embodiment, the first arm is fixedly attached to the support member while the second arm is slidably movable. An adjustment mechanism can be formed on or mated to the proximal end of the second arm to allow movement of the second arm along the support member. The adjustment mechanism can comprise a spring-lock mechanism that is movable between a first, locked position, and a second position wherein the second arm is slidable along the support member. Alternatively, by way of non-limiting example, the adjustment mechanism can comprise threads formed on each of the support member and the second arm such that rotation of the support member is effective to move the second arm with respect to the first arm.

In another embodiment of the present invention, an adjustable guide member is provided having a first member with an elongate support and a first arm mated to one end thereof. The arm preferably extends in a direction transverse to the support and includes a first guide member mated to a distal end thereof. The adjustable guide member further includes a second member having a second arm with a first end adapted to slidably mate with and extend in a direction transverse to the elongate support of the first member. The second arm includes a second guide member mated to a distal end thereof. At least one of the guide members is preferably adapted to receive a tool therethrough.

In other aspects of the present invention, the adjustable guide device can further include a third arm mated to the first guide member and a fourth arm mated to the second guide member. Preferably, the first guide member comprises a frame having a first end mated to the first arm and a second, opposed end mated to the third arm, and the second guide member comprises a frame having a first end mated to the second arm and a second, opposed end mated to the fourth arm. The support member can optionally be movable between a first position, in which it is slidably mated to the first and second arms, and a second position, in which it is slidably mated to the third and fourth arms. The device can also optionally include a second support member mated to the third and fourth arms.

In yet another embodiment of the present invention, a spinal fixation kit is provided including a spinal fixation plate having a proximal portion with at least one bore formed therein for receiving a fixation device effective to mate the proximal portion to a first vertebrae, and a distal portion with at least one bore formed therein for receiving a fixation device effective to mate the distal portion to a second, adjacent vertebrae. The kit further includes a guide device having an elongate support member, a first arm having a proximal end mated to the elongate support member and a distal end with at least one guide member coupled thereto, the guide member being configured for juxtaposition on the proximal portion of the spinal fixation plate, and a second arm having a proximal end mated to the elongate support member and a distal end with at least one guide member coupled thereto, the guide member being configured for juxtaposition on the distal portion of the spinal fixation plate.

Methods for using the devices of the present invention are also provided.

### BRIEF DESCRIPTION OF THE DRAWINGS

The invention will be more fully understood from the following detailed description taken in conjunction with the accompanying drawings, in which:

FIG. 1 is a perspective view of a guide device according to one embodiment of the present invention;

FIG. 2 is a perspective view of the guide device shown in FIG. 1 in an unassembled state;

FIG. 3A is a perspective view of the guide device member portion of the guide device shown in FIG. 1;

FIG. 3B is a perspective view of the distal end of the guide device member shown in FIG. 3A;

FIG. 4 is a perspective view of a guide device according to the present invention having a spinal fixation plate mated thereto;

FIG. 5A is a perspective view of another embodiment of a guide device according to the present invention;

FIG. 5B is a perspective view of the guide member of the guide device shown in FIG. 5A;

FIG. 5C is a cross-sectional view of the guide member shown in FIG. 5B;

FIG. 5D is a top view of the guide member shown in FIG. 5A;

FIG. 6 is a perspective view of another embodiment of a guide device according to the present invention;

FIG. 7 is a perspective view of yet another embodiment of a guide device according to the present invention; and

FIG. 8 is a perspective view of another embodiment of a guide device according to the present invention shown positioned in a patient's spine.

### DETAILED DESCRIPTION OF THE INVENTION

In general, the present invention provides a guide device for use in securing a spinal implant, such as a fixation plate, to a patient's spine. The guide device generally includes a support member having first and second arms mated thereto. Each arm includes a proximal end and a distal end having a guide member with at least one pathway extending therethrough for receiving a tool. One or both arms can be slidably movable along the support member to allow the distance between the arms to be adjusted. In use, the arms can be adjusted to engage a spinal implant and to position the pathways through each guide member in alignment with corresponding bores formed in the implant, thereby providing a fixed entry angle for tools being inserted through the pathways. The guide device can then be used to drill, awl, tap, and insert implants, such as spinal screws, into the vertebral bodies to attach the implant thereto. The guide device is particularly advantageous in that it can function as a guide device, a midline alignment device, as well as a plate inserter for a range of plate sizes. The device further provides a more time efficient and simplified surgical procedure, eliminating several unnecessary steps and instruments typically required to implant a spinal implant, such as a spinal fixation plate.

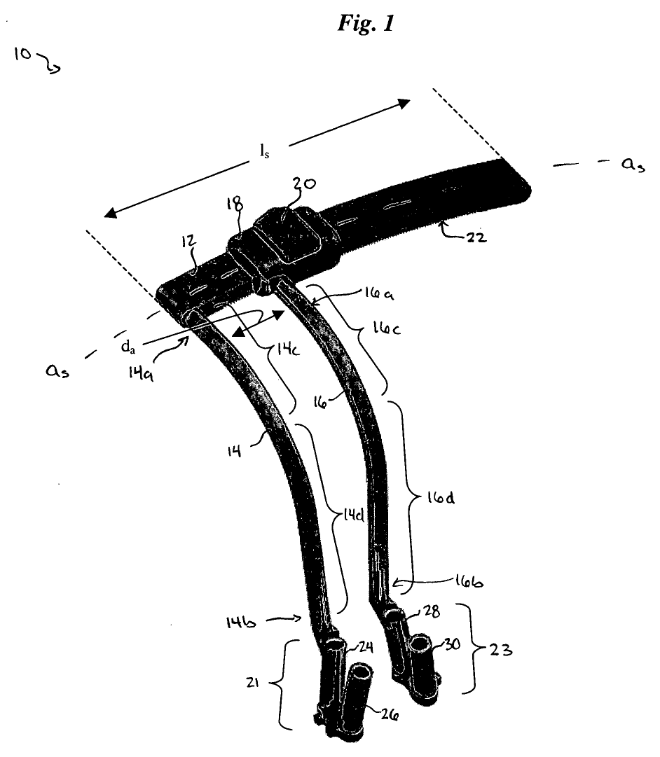

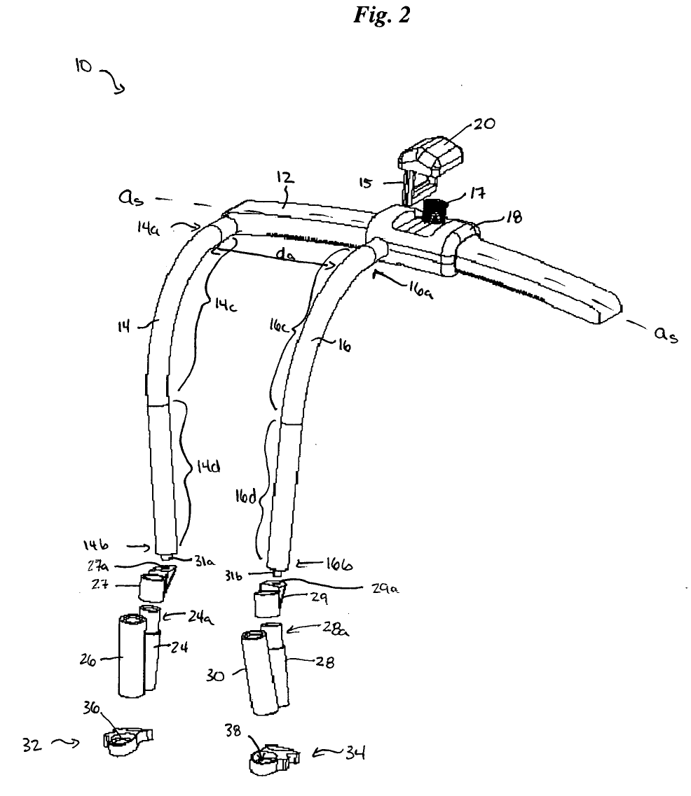

FIGS. 1 and 2 illustrate one embodiment of a guide device 10 according to the present invention. As shown, the guide device 10 includes an elongate support member 12 having first and second arms 14, 16 mated thereto, at least one of which is preferably slidably mated to the support member 12. Each arm 14, 16 includes a guide member 21, 23 mated to the distal end thereof for drilling, tapping, and inserting implants into vertebral bodies. The support member 12 can have a variety of shapes and sizes, but it preferably is an elongate member having a profile that is slightly curved along the longitudinal axis as such that the profile is adapted to match the contour of a patient's spine. The curvature in the support member 12 facilitates the proper positioning of the guide members 21, 23 as the distance between the arms 14, 16 is adjusted, which will be discussed in more detail below. The support member 12 can have a variety of cross-sectional shapes such as, for example, square, circular, oval, rectangular, triangular, etc. The length lₛ of the support member 12 can also vary, but the length lₛ is preferably sufficient to allow the arms 14, 16 to move a distance dₐ apart from one another to engage a spinal fixation plate, as discussed below. In an exemplary embodiment, the length lₛ of the support member 12 is in the range of about 25 mm to 200 mm, and more preferably is about 100 mm to 150 mm. The support member 12 can also be adapted to mate to another support, such as a mounting rack (not shown) used during surgical procedures. A person having ordinary skill in the art will appreciate that the support member 12 can have a variety of configurations.

Still referring to FIGS. 1 and 2, the arms 14, 16 each include a proximal end 14a, 16a mated to the support member 12, and a distal end 14b, 16b. The proximal end 14a, 16a of one or both arms 14, 16 can be slidably mated to the support member 12. Preferably, as shown, one of the arms 14, 16, e.g., the first arm 14, is fixedly mated to one end of the support member 12, and the other arm 14, 16, e.g., the second arm 16, is slidably mated to the support member 12 and movable along the remaining length lₛ of the support member 12. A variety of mating techniques can be used to slidably mate the second arm 16 to the support member. By way of non-limiting example, FIGS. 1 and 2 illustrate a box-like housing 18 formed on or mated to the proximal end of the second arm 16 and having a push-button 20 disposed therein. The box-like housing 18 is adapted to fit around and slidably receive the support member 12, and the push-button 20 is effective to engage the support member 12 to prevent movement of the second arm 16. As shown in FIG. 2, the push-button 20 includes a substantially rectangular or square engagement member 15 that extends distally from the push-button 20. The engagement member 15 slidably receives the support member 12 and is effective to engage a series of ridges 22 formed on the support member 12. The push-button 20 further includes a spring 17 disposed therein for allowing the button 20 to be activated. In use, the spring 17 applies a force onto the push-button 20 to cause the engagement mechanism 15 to grasp the ridges 22 formed on the support member, thereby retaining the arm 16 in the locked position. The arm 16 can be moved by pressing down on the push-button 20 and thereby releasing the engagement mechanism 15 from the ridges 22. The arm 16 is then free to slide along the support member 12 while the button 20 is held in the down position. A person having ordinary skill in the art will appreciate that a virtually any technique can be used to allow slidable movement of the second arm 16 along the support member 12. Moreover, as previously stated, both arms 14, 16 can optionally be movable along the support member 12.

Each arm 14, 16 can have a variety of shapes and sizes, but preferably each arm 14, 16 has a generally elongate shape to allow the distal end 14b, 16b of each arm 14, 16 to be positioned at a surgical site while the support member 12 is positioned outside the surgical field. While the arms 14, 16 can be substantially straight, the arms 14, 16 are preferably curved to prevent the support member 12 from hindering or blocking the surgeon's view of the surgical site. In an exemplary embodiment, each arm 14, 16 includes a proximal portion 14c, 16c that extends in a direction substantially perpendicular to the longitudinal axis as of the support member 12, and a distal portion 14d, 16d that extends in a direction substantially perpendicular to the proximal portion 14c, 16c of the arms 14, 16. The proximal and distal sections 14c, 16c, 14d, 16d can be bent with respect to one another, but they are preferably curved to provide a smooth profile. In an exemplary embodiment, the arms 14, 16 have a shape and size that does not require a large incision to be made in order to use the device. As shown in FIG. 1, for example, the arms 14, 16 have a generally small diameter and are curved slightly toward one another to allow each arm 14, 16 to be inserted through a relatively small incision. Moreover, the distal end of the guide member 21, 23 on each arm 14, 16 is positioned at a distance apart from one another that is greater than the distance between the proximal end of each guide member 21, 23. As a result, the distance required for the guide members 21, 23 to mate to a spinal fixation plate is slightly reduced.

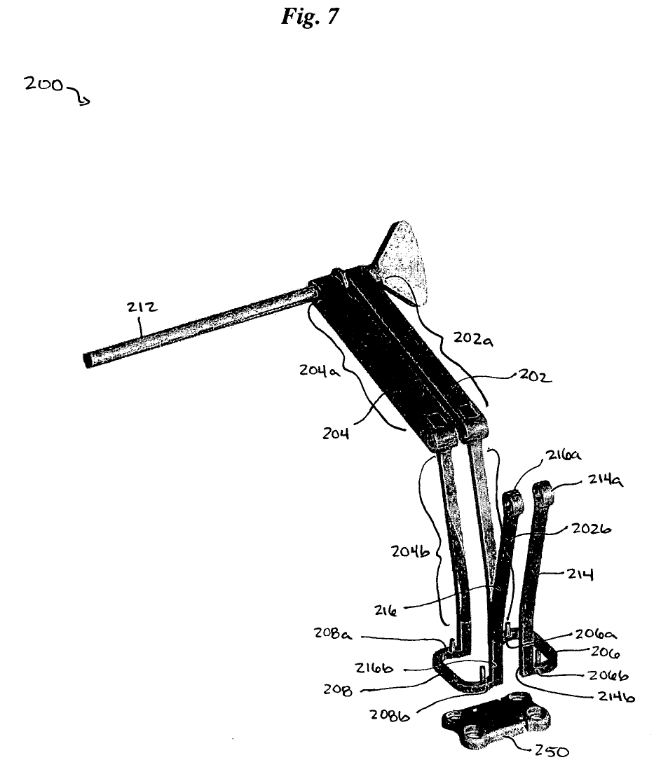

A person having ordinary skill in the art will appreciate that each arm 14, 16 can have virtually any shape and size, and that FIGS. 1 and 2 only illustrate one preferred embodiment. Moreover, while FIGS. 1-4 illustrate a guide device 10 having only two arms 14, 16, the guide 10 can include any number of arms (not shown) to allow the device to used with one or several spinal fixation plates. The guide device can also include additional arms opposed to the first and second arms for allowing the support member to be positioned on one or both sides of a fixation plate being engaged by the guide device 10, as will be described in more detail with respect to FIG. 7.

The distal end 14b, 16b of each arm 14, 16 is preferably adapted to mate to or engage a spinal fixation plate, and can thus can include a guide member 21, 23 formed thereon or mated thereto. Each guide member 21, 23 can have a variety of configurations, but at least one of the guide members 21, 23 preferably includes at least one pathway formed therethrough for receiving a tool, as will be described in more detail below. The arms 14, 16 can be fixedly mated to the guide members 21, 23, or alternatively they can be removably mated to the guide members 21, 23. Moreover, the arms 14, 16 can be mated to any portion of the guide members 21, 23. In one embodiment, each arm 14, 16 can attach to a proximal end of the guide member 21, 23 at an offset position, such that the arms 14, 16 are positioned off to the side of the guide member 21, 23 so as to avoid interference with use of the guide member 21, 23, and to provide better visual access to the surgical site. In an exemplary embodiment, shown in FIG. 2, the distal end 14b, 16b of each arm 14, 16 can mate to an extension member 27, 29, which preferably extends from each guide member 21, 23 in a direction transverse to the arm 14, 16. As is further shown in FIG. 2, the proximal end 24a of barrel 24 is mated to extension member 29, and the proximal end 28a of barrel 28 is mated to extension 27. While virtually any mating technique can be used to mate the arms 14, 16 to the extension members 27, 29, the extension member 27, 29 can include, for example, a bore 27a, 29a formed therein for receiving a corresponding pin member 31a, 31b formed on a distal end 14b, 16b of each arm 14, 16. A person having ordinary skill in the art will appreciate that the arms 14, 16 can be mated to any portion of the guide members 21, 23, and alternatively the arms 14, 16 can be adapted for use in other surgical procedures.

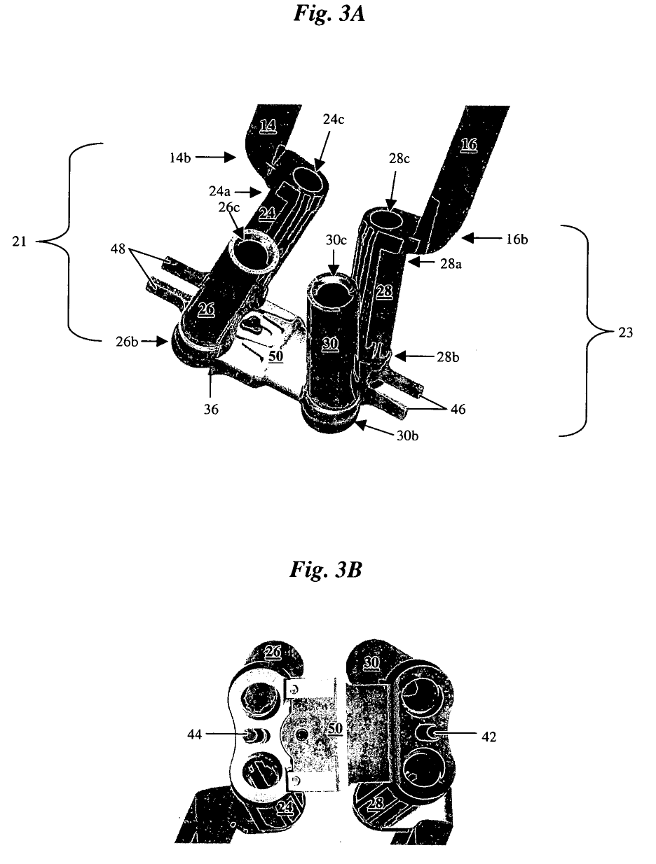

As previously stated, each guide member can have a variety of configurations, but they should include at least one pathway formed therethrough for receiving a tool. FIGS. 3A-3B illustrate one embodiment of guide members 21, 23 having two barrels 24, 26, 28, 30 mated thereto and defining pathways for receiving a tool. The barrels 24, 26, 28, 30 can be removably or fixedly mated to one another, and/or they can be removable from a portion of each guide member 21, 23. Removable barrels 24, 26, 28, 30 are particularly advantageous in that they allow barrels having different lengths to be selected based on the intended use.

Each barrel 24, 26, 28, 30 can have a variety of shapes and sizes, but they should be adapted to receive a tool, such as awl, a drill bit, a fastener, or a driver device. In the embodiment shown in FIGS. 1-3B, each barrel 24, 26, 28, 30 has a generally cylindrical shape and includes a proximal end 24a, 26a, 28a, 30a, a distal end 24b, 26b, 28b, 30b, and an inner lumen 24c, 26c, 28c, 30c extending therebetween. A base plate 32, 34 extends between the distal ends 24b, 26b, 28b, 30b of each set of barrels to mate the barrels 24, 26, 28, 30 to one another, and the proximal end of one of the two barrels, e.g., barrel 24a and barrel 28a, is mated to the distal end 14b, 16b of an arm 14, 16. The base plates 32, 34 that mate the barrels 24, 26, 28, 30 can optionally include bores (not shown) formed therein for removably or fixedly receiving the barrels 24, 26, 28, 30.

The base plates 32, 34 can have a variety of configurations, but preferably each base plate 32, 34, or at least a distal surface of each base plate 32, 34, has a shape adapted to match the contour of a spinal fixation plate. Each base plate 32, 34 should also have a shape and size that results in the alignment of the barrels 24, 26, 28, 30 with corresponding bores formed in a spinal fixation plate being engaged by the guide device.

The barrels 24, 26, 28, 30 are preferably disposed at a predetermined angle a with respect to the base plates 32, 34, or alternatively the base plates 32, 34 have a shape that causes the barrels 24, 26, 28, 30 to be positioned at an angle a with respect to a spinal fixation plate being engaged by the guide device 10. The angle a of each barrel 24, 26, 28, 30 is determinative of the entry angle a of a tool or device being inserted therethrough, and thus the angle a should be set based on the intended use. The angle a of one or more of the barrels 24, 26, 28, 30 can also optionally be adjustable. In an exemplary embodiment, each barrel 24, 26, 28, 30 is positioned so that it is aligned with an axis of a corresponding bore formed in the spinal fixation plate 50 adapted to be engaged by the guide device 10.

Each base plate 32, 34 can also be adapted to engage a spinal fixation plate 50, and thus can include one or more mating elements formed thereon. While a variety of mating elements can be used to mate each guide member 21, 23 to a spinal fixation plate, FIG. 3B illustrates one embodiment of a mating element 42, 44 formed on each base plate 32, 34. As shown, the mating elements each comprise a protrusion or pin member 42, 44 that extends from the distal surface of each base plate 32, 34. The pin members 42, 44 are adapted to extend into corresponding detents or bores formed along the midline of a fixation plate. Upon movement of the arms 14, 16 away from one another, the pin members 42, 44 engage the plate 50. The pin members can optionally be in the form of a hook or similar device effective to grasp the plate. The pin members 42, 44 can also optionally extend at an angle, preferably toward one another, to further facilitate grasping of the fixation plate 50. A person having ordinary skill in the art will appreciate that a variety of techniques can be used to mate the guide device 10 to a spinal fixation plate 50. Moreover, the mating element can be adapted to grasp any portion of a fixation plate. By way of non-limiting example, other suitable mating techniques include a snap-fit engagement, a magnetic engagement, an interference fit, and any other mechanical connection.

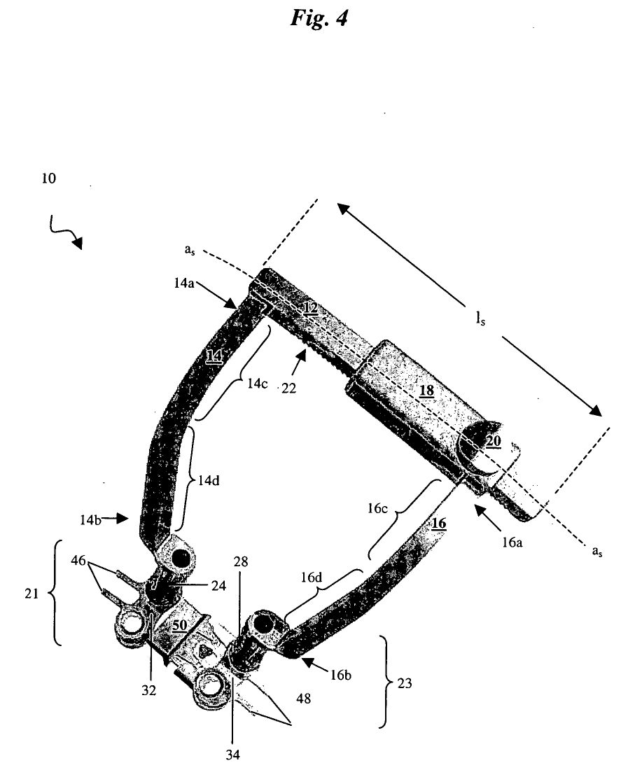

Each plate 32, 34 can also optionally include an alignment feature for aligning the guide device 10 during implantation of a fixation plate 50. While a variety of alignment features can be used, in an exemplary embodiment the alignment feature is a fork member 46, 48, as shown in FIGS. 3A and 4, that extends outwardly from each guide member 21, 23. Typically, during implantation of a spinal fixation plate, Caspar pins are inserted into adjacent vertebral bodies and are used to distract the vertebrae. The Caspar pins can be left in place while the plate is being secured to the vertebrae, thereby allowing the fork-like members 46, 48 on guide device 10 to be placed around the Caspar pins to facilitate positioning of the plate.

In another embodiment, the alignment mechanism can be formed on one or both guide members 21, 23 and can be effective to align the guide member 21, 23 with the endplate of a vertebral body. FIG. 3 illustrates a fin 36 formed on a distal surface of one side of spinal fixation plate 50. A similar type of fin 36 can optionally be formed on one or both of the guide members 21, 23. Preferably, the fin 36 is formed on the guide member 21 that is positioned adjacent the superior endplate, rather than the inferior endplate. In use, the fin 36 abuts the endplate to align the guide members 21, 23 with the adjacent vertebrae.

FIG. 4 illustrates the guide device 10 in use. As shown, the first and second arms 14, 16 can be positioned with respect to one another to grasp a fixation plate 50. A variety of fixation plates 50 can be used with the present invention, including fixation plates having an adjustable size. While the guide device 10 can be adapted to position the guide member 21, 23 at different locations on a fixation plate, preferably one of the guide members, e.g., guide member 21, is positioned on the superior end of a fixation plate, and the other guide member, e.g., guide member 23, is positioned on the inferior end of a fixation plate. This is particularly advantageous in that a fixation plate can be fastened to adjacent vertebrae using a single guide device that does not need to be repositioned during use. An even further advantage is provided where the device includes several arms, as several guide members can be positioned along a length of a patient's spine to fasten one or more fixation plates to one or more adjacent vertebrae without the need to reposition the device during use. As previously described above, the plate 50 can be grasped by positioning the mating element 42, 44 formed on each guide member 21, 23 within corresponding detents or bores formed in the fixation plate 50. The arms 14, 16 are then moved away from one another, by pressing on the push-button 20, to grasp the fixation plate 50. Where a plate having an adjustable length is used, the arms 14, 16 can be moved to adjust the length of the plate, as desired. The support member can optionally include a measurement gauge for setting the length of the fixation plate, if necessary. If provided, and if Caspar pins are used during the surgery, the fork-like members 48, 46 can be placed around the Caspar pins to position the plate 50 with respect to the adjacent vertebrae. One or more of the barrels 24, 26, 28, 30, and/or the bores (not shown) formed in the base plates 32, 34, can be used to drill, awl, tap, and insert tools and implants, such as spinal screws, to secure the fixation plate 50 to the adjacent vertebrae.

A person having ordinary skill in the art will appreciate that while FIGS. 1-4 illustrate arms 14, 16 having guide member 21, 23 with two barrels 24, 26, 28, 30 mated thereto, the device 10 can have a variety of configurations. By way of non-limiting example, only one of the two arms 14, 16 can include a guide member 21, 23 formed thereon, and the guide member 21, 23 can include any number of barrels 24, 26, 28, 30 and/or guide bores formed therein. Alternatively, one or both arms can form the guide member and can include a bore extending therethrough for receiving a tool. Where the arm is curved, the bore preferably extends through the straightened distal portion of the arm.

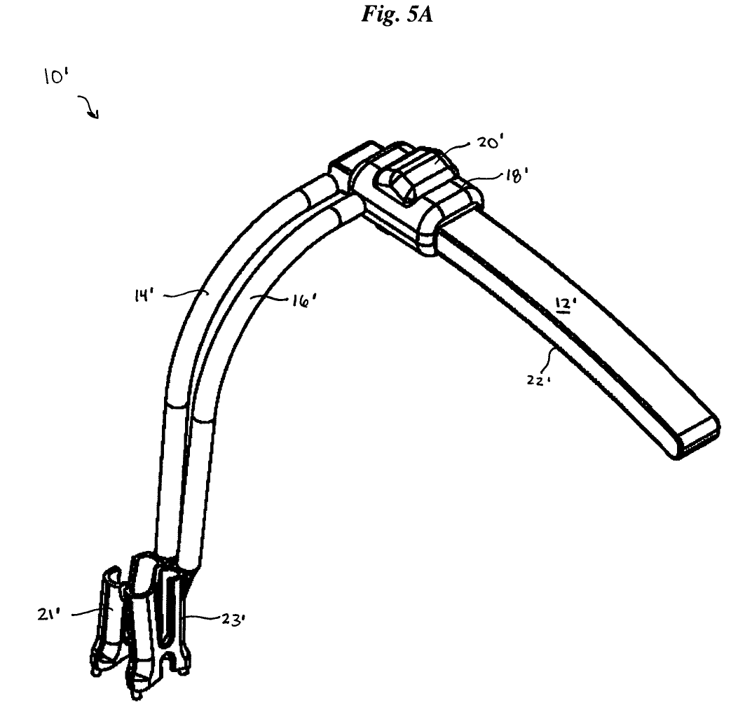

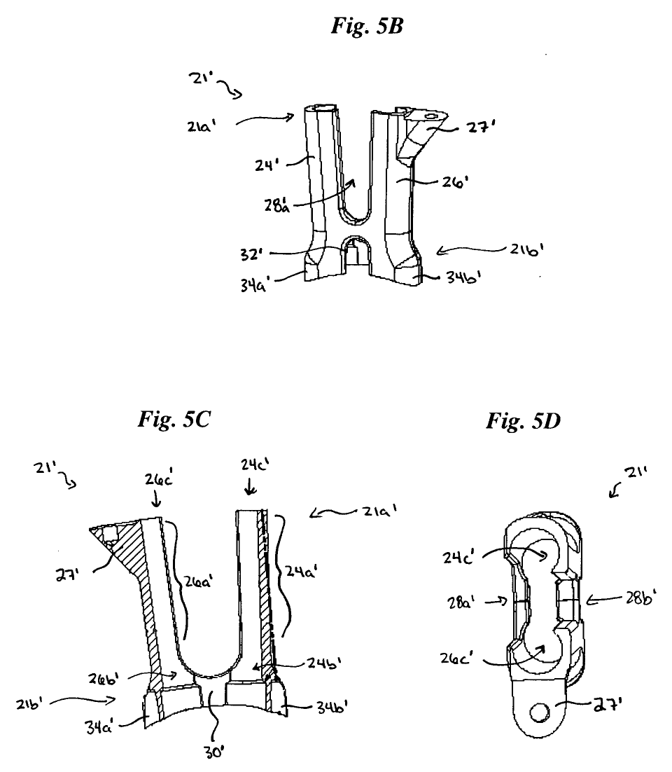

FIGS. 5A-5D illustrate another embodiment of a guide member for use with a guide device in accordance with the present invention. As shown, guide device 10′ is similar to guide device 10, however each guide member 21′, 23′ is in the form of a substantially hollow housing. FIGS. 5B-5D illustrate guide housing 21′ in more detail, and as shown the housing 21′ generally includes first and second pathways 24c′, 26c′ formed therein and extending therethrough between proximal and distal ends 21a′, 21b′ thereof. While the pathways 24c′, 26c′ are formed within a single, hollow lumen that extends through the housing 21′, each pathway 24c′, 26c′ is defined by a substantially semi-cylindrical or C-shaped sidewall 24′, 26′. As a result, each pathway 24c′, 26c′ is configured to receive and guide a tool toward a spinal implant, such as a spinal fixation plate, positioned in relation to the guide member 21′. A person skilled in the art will appreciate that each pathway 24c′, 26c′ can be formed from a separate lumen that extends through the housing 21′, and that the pathways 24c′, 26c′ do not need to be in communication with one another. Moreover, each pathway 24c′, 26c′ can have a variety of shapes and sizes.

As is further illustrated in FIGS. 5B-5D, the housing 21′ can also include one or more cut-out portions or windows formed therein to facilitate visual access to a spinal fixation plate coupled to the guide device 21′. The cut-out portions can be formed anywhere in the housing 21′, but in an exemplary embodiment a first pair of opposed cut-out portions 28a′, 28b′ are formed in opposed sidewalls of the housing 21′ between the first and second pathways 24c′, 26c′. The cut-out portions 28a′, 28b′ extend distally from the proximal end 21a′ of the housing 21′, and they terminate just proximal to the distal end 21b′ of the housing 21′. As a result, the proximal portion 26a′, 24a′ of each pathway 24c′, 26c′ is separated by the cut-out portions 28a′, 28b′, and the distal end 24b′, 26b′ of each pathway 24c′, 26c′ is in communication with one another. As previously mentioned, these cut-out portions 28a′, 28b′ are particularly advantageous in that they provide the surgeon with improved visual access to a spinal plate attached to the guide member 21′, as well as to the tools and devices used in connection with the guide 21′ and spinal fixation plate.

The guide member 21′ can also optionally include a third, distal cut-out portion 32′, shown in FIG. 5B, that is formed adjacent to the distal end 21b′ of the housing 21′. This cut-out portion avoids interference by the guide member 21′ with a temporary fixation pin that is disposed through the spinal fixation plate to temporarily attach the plate to bone. Since temporary fixation pins are typically only placed on opposed ends of the plate, the distal cut-out portions are preferably only formed on one side of each guide member 21′, 23′, such that each guide member 21′, 23′ includes a cut-out portion formed on the outer sides thereof, and the inner sides of the guide members 21′, 23′ that are facing one another do not include distal cut-out portions. A person skilled in the art will appreciate that the shape, size, and location of the distal cut-out portion can vary.

As previously discussed with respect to guide device 10, guide device 10′ is preferably adapted to couple to a spinal implant, and more preferably to a spinal fixation plate. Accordingly, each guide member 21′, 23′ can include a mating element or alignment mechanism formed thereon for engaging or otherwise coupling to a spinal fixation plate. As shown in FIGS. 5A and 5B, the distal end 21b′ of the guide member 21′ has a shape that is adapted to match the shape of a spinal fixation plate, and in particular the distal end 21b′ is substantially concave to seat a convex surface of the plate. The guide member 21′ also includes distally-extending tabs 34a′, 34b′ formed on each sidewall 24′, 26′ that are effective to seat a spinal fixation plate therebetween. The tabs 34a′, 34b′ each preferably have a substantially concave inner surface such that they match the contour of a substantially convex outer surface formed around opposed screw bores formed in a spinal fixation plate. This allows the tabs 34a′, 34b′ to rest against and/or engage opposed outer surfaces of the spinal fixation plate. The tabs 34a′, 34b′ can also optionally be adapted to provide an interference fit with outer edges of the spinal fixation plate to engage the spinal fixation plate. A person skilled in the art will appreciate that the guide member 21′ can include any number of tabs formed on any sidewall thereof, and that each guide member 21′, 23′ can include a variety of other mating elements, including those previously described with respect to guide device 10.

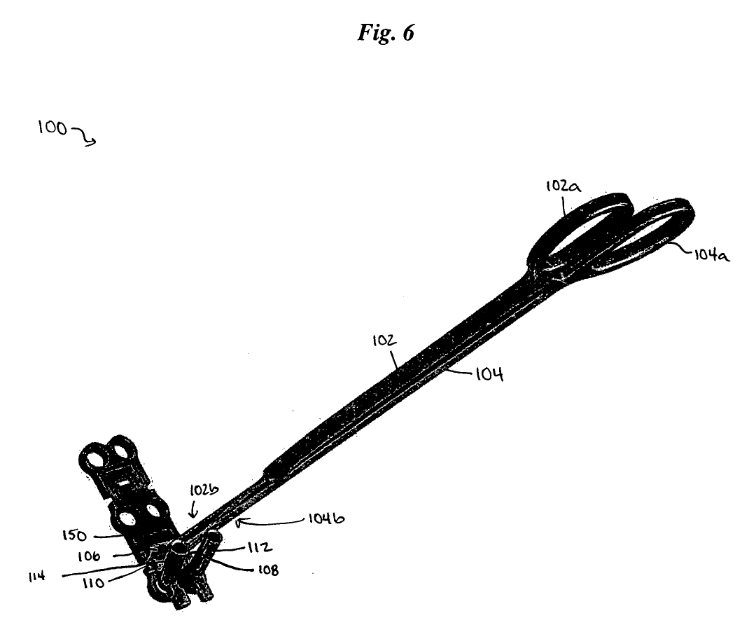

FIG. 6 illustrates another embodiment of a guide device 100 according to the present invention. As shown, the guide device 100 includes first and second arms 102, 104 pivotally coupled to one another and movable between an open position (not shown) and a closed position, as shown. Each arm 102, 104 has a proximal, handle end 102a, 104a and a distal end 102b, 104b. A variety of handle members can be used to grasp the arms 102, 104. As shown, the handle members are in the form of loops 103, 105, similar to scissor handles. The arms 102, 104 can have a variety of configurations, but are preferably generally elongate and are effective to allow movement of the distal ends 102b, 104b toward and away from one another. A first guide member is mated to the distal end 102b of the first arm 102 and has a base plate 106 with a barrel 110 formed thereon for receiving a tool. The second guide member is mated to the distal end 104b of the second arm 104 and also has a base plate 108 and a barrel 112 formed thereon for receiving a tool.

Each base plate 106, 108 can have a variety of configurations, but preferably they are adapted to grasp a spinal fixation plate 150. As shown in FIG. 6, each base plate 106, 108 includes a hook-shaped member 114 (only one hook is shown) that is effective to fit around an edge of the fixation plate 150. In use, the hook members 114 come together to grasp opposed edges of the fixation plate 150 when the first and second arms are positioned in the closed position. Each base plate 106, 108 can also include an alignment mechanism for aligning the fixation plate 150 during implantation. The alignment mechanism is similar to alignment mechanisms 48 and 46 previously described above with respect to FIG. 3A, and can be in the form of a cut-out portion which, when the base plates 106, 108 are combined, form a U-shaped portion that is effective to fit around a Caspar pin.

The barrels 110, 112 can be fixedly attached to or removably mated to each base plate 106, 108, and each base plate 106, 108 can optionally include more than one barrel 110, 112. The barrels 110, 112 are similar to barrels 24, 26, 28, 30 described above with respect to FIGS. 1-4, and thus are preferably positioned at a predetermined angle which is determinative of the entry angle of a tool or implant being introduced into the barrel 110, 112. Alternatively, as was also described above, the base plates 106, 108 themselves can be angled to position the barrels at the desired angle with respect to the fixation plate 150.

In use, the arms 102, 104 are moved to the open position and the base plates 106, 108 are positioned on opposed edges of a fixation plate 150. The arms are then moved to the closed position, thereby causing the base plates 106, 108 to grasp the fixation plate 150. The barrels 110, 112 are thereby aligned with the corresponding bores formed in the fixation plate 150, and can be used to drill, awl, tap, and insert tools and implants, such as spinal screws, to secure the fixation plate 150 to the adjacent vertebrae.

A person having ordinary skill in the art will appreciate that the barrels 110, 112 of the guide device 100 shown in FIG. 6 can each be mated to one arm, e.g., arm 104, and the other arm 102 can merely include a base plate 106 formed thereon. Moreover, the device 100 can include any number of barrels or other guide members formed on one or both arms 102, 104.

FIG. 7 illustrates yet another embodiment of a guide device 200. As shown, the guide device 200 includes a linear support 212 having first and second arms 202, 204 mated thereto. One or both of the arms 202, 204 can be slidably mated to the support, but in an exemplary embodiment one of the arms, e.g., the first arm 202, is fixedly attached to the support 212, and the other arm, e.g., the second arm 204, is slidably mated to the support. While not shown, the second arm 204 can optionally include an adjustment mechanism, similar to adjustment mechanism 20 described above with respect to FIGS. 1, 2, and 4, for allowing the position of the arm 204 to be adjusted along the length of the support 212. In an exemplary embodiment, the adjustment mechanism comprises threads formed on the support member 212 and formed within a lumen extending through the proximal end of the second arm 204. In this embodiment, the first arm 202 should be freely rotatable with respect to the support member 212. In use, rotation of the support member 212 is effective to move the second arm 204 with respect to the first arm 202. In another embodiment (not shown), the rotating knob can be coupled to the second arm 204 and can, upon rotation, be effective to move the second arm 204 along the support 212. A person having ordinary skill in the art will appreciate that virtually any adjustment mechanism can be used to move one or both arms 202, 204 with respect to the support 212.

Each arm 202, 204 can have a variety of configurations, but preferably each arm 202, 204 includes a proximal portion 202a, 204a that extends in a direction substantially transverse to the support member 212, and a distal portion 202b, 204b that extends in a direction substantially transverse to the proximal portion 202a, 204a. The proximal portions 202a, 204a are preferably pivotally mated to the distal portions 202b, 204b to allow the angle of the portions with respect to one another to be adjusted. The distal-most end of each arm 202, 204 is mated to a guide member which is adapted to engage a spinal fixation plate 250. Each guide member can have a variety of configurations, but is preferably a frame 206, 208 having a first end 206a, 208a, and a second, opposed end 206b, 208b. The first end 206a, 208a of each frame 206, 208 is mated to the distal end of the arm 204, 202, respectively.

The device 200 can also include third and fourth arms 214, 216 each having a distal end 214b, 216b mated to the second end 206b, 208b of the frames 206, 208. The third and fourth arms 214, 216 are preferably the same as the distal portion 202b, 204b of the first and second arms 202, 204, however the third and fourth arms 214, 216 are adapted to be positioned on opposed sides of a spinal fixation plate 250 from the first and second arms 202, 204. The third and fourth arms 214, 216 can each optionally include a proximal portion (not shown) mated to a second support member (not shown). Alternatively, the proximal portions 202a, 204a of the first and second arms 204, 202 can be removably mated to the distal portions 202b, 204b, thereby allowing the proximal portion 202a, 204a of the first and second arms 202, 204 to be removed from the distal portion 202b, 204b of the first and second arms 202, 204 and to be attached to the third and fourth arms 214, 216. The use of a movable support member, or two support members, is particularly advantageous in that it allows the surgeon to operate from either side of the patient.

The frame 206, 208 on each arm 202, 204 can be adapted to mate to a spinal fixation plate, and can optionally be adapted to receive one or more barrels (not shown). In an exemplary embodiment, each frame 206, 208 has a shape that is adapted to fit around the outer perimeter of a spinal fixation plate 250. In use, the arms 202, 204 can be moved toward one another along the support 212 to cause the frames 206, 208 to grasp the plate by friction fit. Once engaged, one or more barrels can be attached to the frames 206, 208 to drill, awl, tap, and insert tools and/or implants therethrough to secure the plate to adjacent vertebrae.

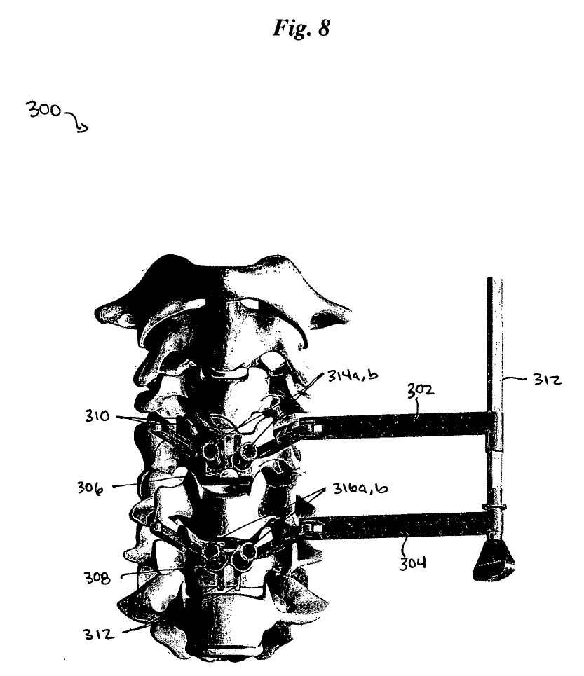

FIG. 8 illustrates yet another embodiment of a guide device 300 positioned along a portion of a patient's spinal column. The guide device 300 is similar to guide device 200, but it does not include frames 206, 208 that are adapted to engage a fixation plate. Rather, each arm 302, 304 includes a guide member having a base plate 306, 308 with two barrels 314a, 314b, 316a, 316b disposed thereon. Each base plate 306, 308 can also include an alignment mechanism, such as a fork-like member 310, 312, formed thereon for aligning the guide members with respect to Caspar pins. A fixation plate can then be aligned and fastened to the vertebrae using one or more spinal screws.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A guide device for use with a spinal implant, comprising:
an elongate support member; and
first and second arms each having a proximal end coupled to the elongate support member and a distal end, at least one of the first and second arms being slidably movable along the support member to allow a distance between the first and second arms to be adjusted, and at least one of the first and second arms including a guide member coupled to a distal end thereof and having at least one pathway formed therein for receiving a tool.

2. The guide device of claim 1, wherein the guide member is adapted to be juxtapositioned on a spinal implant such that the at least one pathway in the guide member is aligned with a corresponding bore formed in the implant.

3. The guide device of claim 1, wherein a first guide member is coupled to the first arm and a second guide member is coupled to the second arm.

4. The guide device of claim 3, wherein each guide member comprises a housing having proximal and distal ends with opposed first and second pathways formed therein and extending therebetween.

5. The guide device of claim 4, wherein the housing is formed from first and second barrels, and the first and second pathways comprise lumens extending through the barrels.

6. The guide device of claim 4, wherein the housing is hollow and the first and second pathways comprise opposed, substantially semi-cylindrical pathways formed within the hollow housing.

7. The guide device of claim 4, wherein at least a portion of each pathway is defined by a substantially elongate, semi-cylindrical sidewall of the housing.

8. The guide device of claim 7, wherein the first arm is mated to a proximal end of one of the semi-cylindrical sidewalls of the first guide, and the second arm is mated to a proximal end of one of the semi-cylindrical sidewalls of the second guide.

9. The guide device of claim 7, wherein a distal end of each semi-cylindrical sidewall extends distally beyond a distal end of the guide member to form opposed tabs that are adapted to seat a spinal fixation plate therebetween.

10. The guide device of claim 9, wherein each tab has a substantially concave inner surface that is adapted to match the contour of a substantially concave outer surface formed around a perimeter of a spinal implant.

11. The guide device of claim 4, wherein at least a portion of each pathway is in communication with one another.

12. The guide device of claim 4, further comprising at least one cut-out portion formed in the housing between the first and second pathways.

13. The guide device of claim 12, wherein the at least one cut-out portion extends from the proximal end of the housing and terminates proximal to the distal end of the housing such that at least a portion of the first and second pathways adjacent the distal end of the housing are in communication with one another.

14. The guide device of claim 1, wherein the elongate support is curved to match the contour of a patient's spine.

15. The guide device of claim 1, wherein the proximal end of each of the first and second arms extends in a direction substantially transverse to the support member.

16. The guide device of claim 1, wherein each of the first and second arms includes a proximal portion and a distal portion, and wherein the proximal portion of each arm extends in a direction substantially transverse to the support member, and wherein the distal portion of each arm extends at an angle with respect to the proximal portion.

17. The guide device of claim 3, wherein the first and second arms are mated to a proximal portion of the first and second guide members.

18. The guide device of claim 3, wherein each guide member has a distal end having a shape adapted to match the contour of a spinal fixation plate.

19. The guide device of claim 3, wherein each guide member includes opposed tabs extending distally therefrom and adapted to engage a spinal fixation plate.

20. The guide device of claim 19, wherein each tab is substantially C-shaped.

21. The guide device of claim 19, wherein the opposed first and second pathways formed in the housing are positioned between the opposed tabs.

22. The guide device of claim 1, wherein the first arm is fixedly mated to the support member and wherein the second arm is slidably mated to the support member.

23. The guide device of claim 22, further comprising an adjustment mechanism formed on the proximal end of the second arm and effective to allow movement of the second arm along the support member.

24. The guide device of claim 23, wherein the adjustment mechanism comprises a spring-lock mechanism that is movable between a first, locked position, and a second position wherein the second arm is slidable along the support member.

25. The guide device of claim 23, wherein the adjustment mechanism comprises corresponding threads formed on each of the support member and the second arm such that rotation of the support member is effective to move the second arm with respect to the first arm.

26. An adjustable guide member for use with a spinal fixation plate, comprising:
a first member having an elongate support and a first arm mated to one end thereof, the arm extending in a direction transverse to the support and including a first guide member mated to a distal end thereof; and
a second member having a second arm with a first end adapted to slidably mate with and extend in a direction transverse to the elongate support of the first member, the second arm having a second guide member mated to a distal end thereof.

27. The adjustable guide device of claim 26, wherein the elongate support of the first member is curved to match the contour of a patient's spine.

28. The adjustable guide device of claim 26, wherein each of the first and second arms includes a proximal portion and a distal portion, and wherein the proximal portion of each arm extends in a direction substantially transverse to the elongate support of the first member, and wherein the distal portion of each arm extends at an angle with respect to the proximal portion.

29. The adjustable guide device of claim 28, wherein first and second guide members are formed integrally with the distal portion of the first and second arms, and wherein the first and second guide members comprise a bore extending through the distal portion of each of the first and second arms.

30. The adjustable guide device of claim 26, wherein at least one of the first and second guide members includes a pathway formed therein for receiving a tool therethrough.

31. The adjustable guide device of claim 30, wherein each guide member comprises a housing having proximal and distal ends with opposed first and second pathways formed therein and extending therebetween.

32. The adjustable guide device of claim 31, wherein the housing is formed from first and second barrels, and the first and second pathways comprise lumens extending through the barrels.

33. The adjustable guide device of claim 31, wherein at least a portion of each pathway is defined by a substantially elongate, semi-cylindrical sidewall.

34. The adjustable guide device of claim 33, wherein the first arm is mated to a proximal end of one of the semi-cylindrical sidewalls of the first guide, and the second arm is mated to a proximal end of one of the semi-cylindrical sidewalls of the second guide.

35. The adjustable guide device of claim 33, wherein a distal end of each semi-cylindrical sidewall extends distally beyond a distal end of the guide member to form opposed tabs that are adapted to seat a spinal fixation plate therebetween.

36. The adjustable guide device of claim 31, wherein at least a portion of each pathway is in communication with one another.

37. The adjustable guide device of claim 31, further comprising at least one cut-out portion formed in opposed walls of the housing between the first and second pathways.

38. The adjustable guide device of claim 37, wherein the at least one cut-out portion extends from the proximal end of the housing and terminates proximal to the distal end of the housing such that at least a portion of the first and second pathways adjacent the distal end of the housing are in communication with one another.

39. The adjustable guide device of claim 26, wherein the guide member on each arm comprises:
a first barrel having a distal end and a proximal end mated to the distal end of the arm; and
a base plate mated to the distal end of the first barrel.

40. The adjustable guide device of claim 39, wherein the guide member on each arm further comprises a second barrel mated to the base plate.

41. The adjustable guide device of claim 39, wherein each base plate includes a mating element formed thereon for mating with a spinal fixation plate.

42. The adjustable guide device of claim 39, wherein each base plate has a shape adapted to match the contour of a spinal fixation plate.

43. The adjustable guide device of claim 41, wherein the mating element is formed on a distal surface of each base plate.

44. The adjustable guide device of claim 43, wherein each mating element comprises a protrusion extending distally from the distal surface of the base plate and adapted to engage a spinal fixation plate.

45. The adjustable guide device of claim 26, further comprising an adjustment mechanism formed on the second member and effective to allow movement of the second member along the elongate support of the first member.

46. The adjustable guide device of claim 45, wherein the adjustment mechanism is movable between a first, locked position, and a second position wherein the second support member is slidable along the first support member.

47. The adjustable guide device of claim 45, wherein the elongate support is rotatably mated to the first arm and the adjustment mechanism comprises corresponding threads formed on each of the elongate support and the second arm such that rotation of the elongate support is effective to move the second arm with respect to the first arm.

48. The adjustable guide device of claim 26, wherein the first arm is slidably mated to the elongate support.

49. The adjustable guide device of claim 26, wherein the first and second guide members each comprise a frame having a shape adapted to match the contour of a spinal fixation plate.

50. The adjustable guide device of claim 49, wherein the frame of at least one of the first and second guide members is adapted to removably receive one or more barrels.

51. The adjustable guide device of claim 26, further comprising a third arm mated to the first guide member and a fourth arm mated to the second guide member.

52. The adjustable guide device of claim 51, wherein the first guide member comprises a frame having a first end mated to the first arm and a second, opposed end mated to the third arm, and wherein the second guide member comprises a frame having a first end mated to the second arm and a second, opposed end mated to the fourth arm.

53. The adjustable guide device of claim 52, wherein the support member is movable between a first position, in which it is mated to the first and second arms, and a second position, in which it is mated to the third and fourth arms.

54. The adjustable guide device of claim 52, further comprising a second support member slidably mated to the third and fourth arms.

55. A spinal fixation kit, comprising:
a spinal fixation plate having
a proximal portion with at least one bore formed therein for receiving a fixation device effective to mate the proximal portion to a first vertebrae, and
a distal portion with at least one bore formed therein for receiving a fixation device effective to mate the distal portion to a second, adjacent vertebrae; and
a guide device having
a first arm with a guide member coupled to a distal end thereof and configured for juxtaposition on the proximal portion of the spinal fixation plate, and
a second arm with guide member coupled a distal end thereof and configured for juxtaposition on the distal portion of the spinal fixation plate.

56. The spinal fixation kit of claim 55, wherein a proximal end of each arm is coupled to an elongate support member.

57. The spinal fixation kit of claim 56, wherein the elongate support member is curved to match the contour of a patient's spine.

58. The spinal fixation kit of claim 56, wherein each of the first and second arms includes a proximal portion and a distal portion, and wherein the proximal portion of each arm extends in a direction substantially transverse to the support member, and wherein the distal portion of each arm extends at an angle with respect to the proximal portion.

59. The spinal fixation kit of claim 55, wherein at least one of the first and second guide members includes a pathway formed therein for receiving a tool therethrough.

60. The spinal fixation kit of claim 59, wherein each guide member comprises a housing having proximal and distal ends with opposed first and second pathways formed therein and extending therebetween.

61. The spinal fixation kit of claim 60, wherein at least a portion of each pathway is defined by a substantially elongate, semi-cylindrical sidewall.

62. The spinal fixation kit of claim 61, wherein the first arm is mated to a proximal end of one of the semi-cylindrical sidewalls of the first guide, and the second arm is mated to a proximal end of one of the semi-cylindrical sidewalls of the second guide.

63. The spinal fixation kit of claim 61, wherein a distal end of each semi-cylindrical sidewall extends distally from a distal end of the guide member to form opposed tabs that are adapted to seat a spinal fixation plate therebetween.

64. The spinal fixation kit of claim 60, wherein at least a portion of each pathway is in communication with one another.

65. The spinal fixation kit of claim 60, further comprising at least one cut-out portion formed in the housing between the first and second pathways.

66. The spinal fixation kit of claim 65, wherein the at least one cut-out portion extends distally from the proximal end of the housing such that at least a portion of the first and second pathways are in communication with one another.

67. The spinal fixation kit of claim 55, wherein the guide member on at least one of the arms includes at least one barrel.

68. The spinal fixation kit of claim 67, wherein each of the at least one barrels is disposed at an angle with respect to the spinal fixation plate.

69. The spinal fixation kit of claim 67, wherein the angle of at least one of the barrels is adjustable.

70. The spinal fixation kit of claim 55, wherein the guide member on each arm comprises:
a first barrel having a distal end and a proximal end mated to the distal end of the arm; and
a base plate mated to the distal end of the first barrel.

71. The spinal fixation kit of claim 70, further comprising a second barrel mated to the base plate.

72. The spinal fixation kit of claim 70, wherein each base plate has a shape adapted to match the contour at least a portion of the spinal fixation plate.

73. The spinal fixation kit of claim 70, wherein each base plate includes a mating element formed thereon for mating with the spinal fixation plate.

74. The spinal fixation kit of claim 73, wherein the mating element is formed on a distal surface of each base plate.

75. The spinal fixation kit of claim 74, wherein each mating element comprises a protrusion that is oriented at an angle so that they are effective to grasp the spinal fixation plate when the arms are moved either away from or toward one another.

76. The spinal fixation kit of claim 56, further comprising an adjustment mechanism formed on the proximal end of the second arm and effective to allow movement of the second arm along the support member.

77. The spinal fixation kit of claim 76, wherein the adjustment mechanism is movable between a first, locked position, and a second position wherein the second arm is slidable along the support member.

78. The spinal fixation kit of claim 76, wherein the adjustment mechanism comprises threads that are effective to mate with corresponding threads formed on the support member such that rotation of the support member is effective to move the second arm with respect to the first arm.

79. A guide device and spinal fixation plate inserter, comprising:
first and second arms pivotally coupled to one another and movable between an open position and a closed position, each arm having a proximal, handle end and a distal end; and
a first guide member mated to the distal end of the first arm and having at least one pathway formed thereon for receiving a tool, and a second guide member mated to the distal end of the second arm and having at least one pathway formed thereon for receiving a tool, the first and second guide members being effective to engage a spinal fixation plate when the first and second arms are positioned in the closed position.

80. The guide device and spinal fixation plate inserter of claim 79, wherein the guide member mated to each of the first and second arms comprises a substantially hollow housing having first and second pathways extending therethrough.

81. The guide device and spinal fixation plate inserter of claim 80, wherein a distal end of each housing has a shape that is adapted to couple to a spinal fixation plate.

82. The guide device and spinal fixation plate inserter of claim 80, wherein a distal end of each housing includes a mating element formed on a distal surface thereof for mating with a spinal fixation plate.

83. The guide device and spinal fixation plate inserter of claim 82, wherein each mating element comprises a tab extending distally from the distal surface of the housing and adapted to engage a spinal fixation plate.

84. The guide device and spinal fixation plate inserter of claim 82, wherein each mating element comprises a protrusion extending distally from the distal surface of the housing and adapted to engage a spinal fixation plate.

85. The guide device and spinal fixation plate inserter of claim 80, further comprising a U-shaped slot formed in the housing for receiving a pin member.

86. A guide device for use with a spinal fixation plate, comprising:
first and second guide members adapted to be juxtaposition on proximal and distal portions of a spinal fixation plate, each guide member including at least one pathway formed therein for receiving a tool, and opposed tabs extending distally therefrom and having a shape adapted to match the contour of at least a portion of a perimeter of the spinal fixation plate.

87. The guide device of claim 86, wherein each tab is substantially C-shaped.

88. The guide device of claim 86, wherein each guide member further includes at least one cut-out portion formed therein for providing visual access to a spinal fixation plate coupled thereto.

89. The guide device of claim 88, wherein each guide member includes first and second pathways extending therethrough from a proximal end to a distal end, and wherein the at least one cut-out portion is formed between the first and second pathways.

90. The guide device of claim 89, wherein each guide member includes opposed cut-out portions formed in opposed sidewalls of the guide member between the first and second pathways.

91. A guide device for use with a spinal fixation plate, comprising:
first and second arms movable with respect to one another, each arm including a proximal end and a distal end; and
a first guide member coupled to the distal end of the first arm, and a second guide member coupled to the distal end of the second arm, each guide member having opposed cut-out portions formed therein and adapted to provide visual access to a spinal fixation plate coupled thereto.

92. The guide device of claim 91, wherein the opposed cut-out portions extend from a proximal end of each guide member and terminate just proximal to a distal end of each guide member.

93. The guide device of claim 91, wherein the opposed cut-out portions are formed in opposed sidewalls of each guide member between opposed pathways extending through the guide member.
